(19) United States
(12) Patent Application Publication
(10) Pub. No.: US 2009/0232895 A1
(43) Pub. Date: Sep. 17, 2009

(54) PROCEDURE AND DEVICES FOR THE CONTROLLED OBTAINING OF DRY SALINE AEROSOLS WITH THERAPEUTIC EFFECT

(76) Inventor: Costantin Pascu, Buzau (RO)

Correspondence Address: John Alumit, 16830 Ventura Blvd. Suite 360, Encino, CA, 91436, US

(21) Appl. No.: 12/466,285

(22) Filed: May 14, 2009

(30) Foreign Application Priority Data
Nov. 14, 2006 (RO) PCT/RO2007/000023

(51) Int. Cl. A61K 9/14; A61K 33/14; A61K 33/00; A61K 33/04; A61P 11/00

(52) U.S. Cl. 424/489; 424/679; 424/680; 424/715; 424/709

(57) ABSTRACT

The patent refers to a procedure and devices for the generation of dry aerosols, continuously, by mechanic self-erosion, by average stirring in air or air-oxygen feed, of some crystals with special structure obtained by controlled crystallization processes. There can be also used other salts known to have physiological effects or mixtures of saline crystals with capacities close to the micro-particle generation. The devices can be for human or veterinary individual use, in intensive therapies or for producing aerosols with therapeutic or preventive effect in public rooms.

### CROSS-REFERENCE TO RELATED APPLICATION

This application claims the benefit of the priority filing date in PCT/RO2007/000023 referenced in WIPO Publication WO/2008/060173. The earliest priority date claimed is Nov. 14, 2006.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention refers to a procedure and devices for producing dry saline aerosols having various concentrations and chemical compositions, for use in different therapeutic procedures and for the prevention of respiratory tract affections.

Procedures involving aerosols in the treatment of various respiratory affections are well known and much used. Respiratory affections constitute an average of over 50% of labor incapacity cases. Sodium chloride aerosols can enter the respiratory tract up to the level of the small bronchia, causing the mucousciliary's transport to activate. Through phenomena connected with the recovery of normal osmolarity, the lung's functional capacity improves with favorable effects on the general state of health.

Saline aerosols achieve a general Invigoration of the respiratory tract with a bronchi and bronchi-dilator anti-inflammatory effect.

The therapeutic effect of salt mines in the treatment of asthma and other allergic and inflammatory affections of the respiratory tract are well known. This is due to the natural saline aerosols existing in salt mines following contact of the air with the salt mass.

Also well known are the respiratory tract treatment methods with marine aerosols produced by the air's contact with salt-water waves. Treatments with natural aerosols can be done only in location where they are produced, making it necessary for persons with respiratory problems to go to such locations. The treatments are influenced by environmental conditions, depending on the location of the treatment place, the season, and the weather.

Treatments with aerosols produced by salt solution spraying are carried out in specialized medical offices. Patent Fr 2864448 presents a device for cleaning the nostrils by spraying a sodium hydrogen carbonate.

The aerosols artificially produced by spraying saline solutions are used in specialized medical offices for periodical treatments of persons with respiratory problems. For example, Patent GB 1198787 presents a device for spraying saline solutions with air and U.S. Pat. No. 6,127,429 presents a device for spraying saline solutions with ultrasounds. Permanent salinisation systems for the workplace or home cannot be made due to the corrosive and super-humidification effect produced by this saline solution on the devices that house the solution.

Halo therapy consists of supplying a dry sodium chloride aerosol under 5 microns in the breathing air. The existing devices that distribute pre-encapsulated powder in the breathing air face the challenge of ensuring a very small grading of under 5 microns, without the powder concentration as well as the supply of powders in very small quantities exceeding 1 mg/mc of air.

All of these methods are generally used for treatment, and only persons living or working in locations where the saline aerosols exist or are produced can benefit from the preventive effect of the respiratory affections.

A device for respiratory therapies, called salt pipe, is on the market. It uses rock salt grains placed in a tank that is periodically stirred, and the air inside is inhaled with the help of a mouthpiece. The disadvantage of the device is the weak generation of micron particles due to the strength of the salt grains as well as the forming of powders over 10 microns that are not useful in respiratory therapies but can generate problems to persons with severe salt restrictions. The use of other types of salts, with known physiological effects, has not been exploited.

Patents Ro-117126 and Ro-118229 present procedures and devices that achieve air saline treatment through forced air contact with saline filters with a micro-crystallized structure deposited on plates or as porous tri-dimensional structures used as an adjuvant in respiratory therapies. The procedure is disadvantaged by the small quantity of aerosols that is released, which greatly reduces therapeutic efficiency.

The patent applications RO A 01024/2003 and RO A 00888/2004 refer to a procedure and devices for generating dry saline aerosols basically through vibration, stirring or fluidization of salt grains in an air feed. There is a disadvantage in using these devices. The ionic structure of NaCl crystals causes the formation of small quantities of micro particles that drastically decrease when humidity increases; they also decrease when the usage period of the devices increases. With a more energetic fluidization, particles over 10 microns are produced, and said procedures and devices do not control the release of these particles from salt mixtures.

The procedure for dry aerosol generation according to this invention eliminates the above-mentioned disadvantages in that it uses crystals of 0.1-5 mm with a special structure obtained by crystal controlled shaping which ensures the controlled emission of saline micro-particles. The emission can also be significantly influenced by heat treatment of the crystals and by obtaining crystals through crystallization in salt mixture solutions, whereby, in a first stage, the aerosols as micron dry particles (0.3-5 microns) are continuously produced by mechanic self-erosion due to continuous moderate stirring in an air or air-oxygen feed, and in a second stage, the produced micro-particles are carried off in an air feed. The emission/generation of saline micro-particles is assured for various and controllable chemical compositions and quantities, for time periods that, depending on usage conditions, can vary from minutes to hours. Behavioral control of micro particles is also achieved in regards to humidity in the air, without producing powder. The saline crystals are obtained out of one or more salts by crystallization under controlled conditions, by selecting some grading fractions. Various salts can be used known to have physiological effects such as sulphates, bicarbonates, iodides, chlorides or mixtures of saline crystals with a capacity for generating micro-particles such as NaCl with a controlled structure and NaHCO₃ in various volumetric ratios. Also assured is the ability for obtaining dry aerosols having various concentrations and chemical compositions for use in diversified therapeutic and preventive procedures.

The device for generating dry aerosols, with therapeutic effect, by moderate stirring in air or air-oxygen feed involves placing some grains or grain mixtures with controlled microstructure and composition, having dimensions of 0.1-5 mm, preferably 0.1-2 mm, at the lower part of a cylindrical filtering cartridge with a diameter of 20-15 mm (preferably 30-60 mm), then through an air feed with an adjustable flow of 1-30 l/min, which passes through a lower pipe fitting with a 3-10 mm diameter (preferably 3-6 mm) equipped with a sieve, assuring moderate stirring of the grain layer, while over another sieve the air feed passes through a three-dimensional spongy structure made of saline grains that assure the supplementary treatment by ionization and filtration, of the air that is sent, when enriched with dry aerosols, to a breathing mask or directly to the environment.

The procedure and the devices can be used in the prevention and individual treatment of various chronically or acute affections of the upper or lower respiratory tract of humans and animals, such as sinusitis, laryngitis, COPD, asthma, and bronchial inflammation, as well as various respiratory infections such as colds, flu, prophylactic and curative therapies in various respiratory affections. They can be used for individual use in intensive therapy, some respiratory affections, and in therapeutic procedures in post surgery recovery. The invention assures small concentrations for long periods of administration, or big concentrations for quick treatments, and for special uses such as in therapy for horses, or salt mixtures such as sodium hydrogen carbonate and magnesium sulphate for complex treatment on the respiratory epithelium.

By applying the procedure and the devices, according to the invention, the following advantages are realized:

Chemical, quantitative and grading controlled generation of dry saline micro-particles;

Generation of particles under 5 microns

Major generation in mass, in the range of 1-5 microns

Generation of particles in big quantities for quick use;

Generation of particles in small quantities for long term use

Endurance of particle generation in humidity environments;

Generation over various periods of time

Generation of mixtures of saline particles with controlled composition.

### SUMMARY

The procedure for dry aerosol generation according to this invention eliminates numerous disadvantages in the prior art, such as assuring very small grading of micro particles under 5 microns without powder concentration, weak generation of micron particles due to the great strength of salt grains, small quantity of aerosols released, and decreased micro particles due to humidity, in that it uses crystals of 0.1-5 mm with a special structure obtained by crystal controlled shaping which ensures the controlled emission of saline micro-particles. The emission can also be significantly influenced by heat treatment of the crystals and by obtaining crystals through crystallization in salt mixture solutions, whereby, in a first stage, the aerosols as micron dry particles (0.3-5 microns) are continuously produced by mechanic self-erosion due to continuous moderate stirring in an air or air-oxygen feed, and in a second stage, the produced micro-particles are carried off in an air feed. The emission/generation of saline micro-particles is assured for various and controllable chemical compositions and quantities, for time periods that, depending on usage conditions, can vary from minutes to hours. Behavioral control of micro particles is also achieved in regards to humidity in the air, without producing powder. The saline crystals are obtained out of one or more salts by crystallization under controlled conditions, by selecting some grading fractions. Various salts can be used known to have physiological effects such as sulphates, bicarbonates, iodides, chlorides or mixtures of saline crystals with a capacity for generating micro-particles such as NaCl with a controlled structure and NaHCO₃ in various volumetric ratios. Also assured is the ability for obtaining dry aerosols having various concentrations and chemical compositions for use in diversified therapeutic and preventive procedures.

The device for generating dry aerosols, with therapeutic effect, by moderate stirring in air or air-oxygen feed involves placing some grains or grain mixtures with controlled microstructure and composition, having dimensions of 0.1-5 mm, preferably 0.1-2 mm, at the lower part of a cylindrical filtering cartridge with a diameter of 20-15 mm (preferably 30-60 mm), then through an air feed with an adjustable flow of 1-30 l/min, which passes through a lower pipe fitting with a 3-10 mm diameter (preferably 3-6 mm) equipped with a sieve, assuring moderate stirring of the grain layer, while over another sieve the air feed passes through a three-dimensional spongy structure made of saline grains that assure the supplementary treatment by ionization and filtration, of the air that is sent, when enriched with dry aerosols, to a breathing mask or directly to the environment.

The procedure and the devices can be used in the prevention and individual treatment of various chronically or acute affections of the upper or lower respiratory tract of humans and animals, such as sinusitis, laryngitis, COPD, asthma, and bronchial inflammation, as well as various respiratory infections such as colds, flu, prophylactic and curative therapies in various respiratory affections. They can be used for individual use in intensive therapy, some respiratory affections, and in therapeutic procedures in post surgery recovery. The invention assures small concentrations for long periods of administration, or big concentrations for quick treatments, and for special uses such as in therapy for horses, or salt mixtures such as sodium hydrogen carbonate and magnesium sulphate for complex treatment on the respiratory epithelium.

### BRIEF DESCRIPTION OF THE FIGURES

FIG. 1 is a scheme of a device for the quantitative and grading controlled generation of dry saline micro-particles.

### DETAILED DESCRIPTION

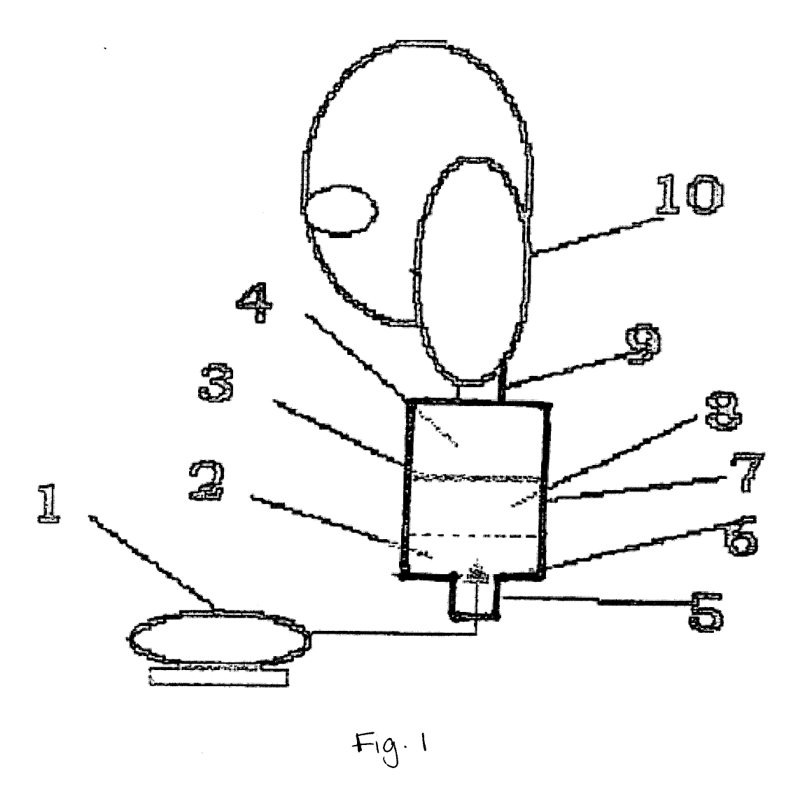

Four examples of carrying out the invention, in connection with FIG. 1

### Example 1

According to patent Ro 11202i, crystals are obtained out of rock salt for food usage. After drying for 2 hours at 70° C., the fraction 100-350 microns of crystals is grinded and separated by screening. 15 cm³ of such crystals are obtained and placed in a device according to FIG. 1. By stirring in an air feed at a flow rate of 3.5 l/min aerosols are produced that are measured with a laser counter device for particles between 0.3-5 microns, for a total number of 10-12 million particles/cubic meter.

Significant therapeutic action is noted in COPD chronic affections, chronic sinusitis, by 30-60 minute daily inhalations.

15 cm³ of crystals obtained in this manner are supplementary treated at 165° C. for 2 hours, then placed in a device according to FIG. 1. By stirring in an air feed of 3.5 l/min aerosols are produced that are measured with a laser counter for particles between 0.3-5 microns, at a total number of 22-25 million particles/cubic meter.

Significant therapeutic action is noted in COPD chronic affections, chronic sinusitis, by 10-15 minute daily inhalations.

In both situations there are no marked out particles over 10 microns at continuous usage of over 50 hours.

Out of the initial rock salt, after drying for 2 hours at 70° C., the fraction 100-350 microns is grinded and separated. 15 cm³ of crystals obtained in this manner are placed in a device according to FIG. 1. By stirring in an air feed at a flow rate of 3.5 l/min aerosols are produced for a total number of 1-1.5 million particles/cubic meter.

15 cm³ of crystals obtained in this manner are supplementary treated at 165° C. for 2 hours, then placed in a device according to FIG. 1. By stirring in an air feed of 3.5 l/min aerosols are produced that are measured with a laser counter for particles between 0.3-5 microns, at a total number of 1.5-2 million particles/cubic meter.

### Example 2

Crystals are obtained by controlled crystallization according to patent IRo 112025, out of a saturated NaCl—Na₂SO₄ solution at a ratio of 10/1. After drying for 2 hours at 70° C., the crystals are grinded and the fraction 100-350 microns is separated. 15 cm³ of crystals are obtained in this manner, and are placed in a device according to FIG. 1. By stirring in an air feed at a flow rate of 3.5 l/min aerosols are produced that are measured for a total number of over 80 million particles/cubic meter. These can be used in respiratory therapies for horses. They can also be used as systems with inhalation for 1-3 minutes in acute or chronic human breathing problems.

### Example 3

Salt crystals are obtained according to example 1. A fraction 100-350 microns are supplementary heat treated at 165° C. for 2 hours. According to the same example, sodium hydrogen carbonate crystals are obtained at a fraction 100-350 microns. The two types of crystals are mixed in the volumetric ratio of NaCl/NaHCO₃ at 3/1.15 cm³ of crystals are placed in a device according to FIG. 1. By stirring in an air feed of 3.5 l/min, aerosols are produced that are measured at a total number of 25-27 million particles/cubic meter. These have decongesting and anti-inflammatory effect marked out at inhalations in ENT problems.

### Example 4

The device of quantitative and grading controlled generation of dry saline micro-particles according to FIG. 1 is comprised of a plastic cartridge 7 with a diameter of 20-150 mm, preferably 30-40 mm and a height of 2-4 times the diameter, the cartridge 7 having two separate compartments at half the distance with plastic perforated sieves 3 between which there is a filtering material (not shown) and in the lower part having an air inlet connection S with a diameter of 3-10 mm, preferably 3-6 mm, equipped with a sieve 6 on part of the cartridge, and in the upper part, the cartridge has a cover equipped with a connection that allows emission to the environment and assures the connection 9 to a mask 10 or to a flexible tube (not shown) that goes to a mask. Between the intermediary sieve and the cartridge cover there are layers 4 made by salt extruded granules with an average granulation of 4-6 mm, and on the lower part of the cartridge, the salt grains 2 are introduced with a controlled structure and composition. By an air or air-oxygen feed passing with an adjustable flow between 1-301/min, preferably between 2-10 l/min, the grains are stirred in a moderate movement generating particles, out of which those over 10 microns are detained when passing through the filter between the intermediary sieves and by the upper grain layer 4.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, paragraph 6. In particular, the use of “step of” in the claims herein is not intended to invoke the provisions of 35 U.S.C. § 112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. A procedure for the generation of dry aerosols with therapeutic effect, characterised by the fact that the aerosols of dry saline micro-particles have a quantitative, grading and chemical controlled composition.

2. A procedure for generating dry aerosols, with therapeutic effect, according to claim 1, characterised by the fact that the aerosols are obtained out of saline crystals of NaCl, KCl, NaHCO₃, Na₂SO₄, MgSO₄ with dimensions of 0.1-5 mm, uniform, combined or mixtures of saline crystals, crystals having controlled manufactured internal structures.

3. A procedure for the generation of dry aerosols with therapeutic effect according to claim 1, characterised by the fact that saline crystals are obtained by controlled crystallization processes, followed by drying, grinding and separation of various fractions, preferably between 100-1000 microns, and supplementary heat treated at 50-250° C. for a minimum of 2 hours; the crystals can be also obtained by the combined crystallization of more salts such as NaCl, KCl, NaHCO₃, Na₂SO₄, MgSO₄, in various proportions.

4. A procedure for the generation of dry aerosols with therapeutic effect according to claim 2, characterised by the fact that saline crystals are obtained by controlled crystallization processes, followed by drying, grinding and separation of various fractions, preferably between 100-1000 microns, and supplementary heat treated at 50-250° C. for a minimum of 2 hours; the crystals can be also obtained by the combined crystallization of more salts such as NaCl, KCl, NaHCO₃, Na₂SO₄, MgSO₄, in various proportions.

5. A procedure for the generation of dry aerosols with therapeutic effect, according to claim 1, characterised by the fact that the aerosols obtained with grain dimensions of 0.3-5 microns and a total number of generated particles of 2-100 mil/mc are used in therapies of short or long duration, in acute or chronic respiratory problems or in human or veterinary problems, as well as in preventive actions for the respiratory diseases, individually or for groups of patients.

6. A procedure for the generation of dry aerosols with therapeutic effect, according to claim 2, characterised by the fact that the aerosols obtained with grain dimensions of 0.3-5 microns and a total number of generated particles of 2-100 mil/mc are used in therapies of short or long duration, in acute or chronic respiratory problems or in human or veterinary problems, as well as in preventive actions for the respiratory diseases, individually or for groups of patients.

7. A procedure for the generation of dry aerosols with therapeutic effect, according to claim 3, characterised by the fact that the aerosols obtained with grain dimensions of 0.3-5 microns and a total number of generated particles of 2-100 mil/mc are used in therapies of short or long duration, in acute or chronic respiratory problems or in human or veterinary problems, as well as in preventive actions for the respiratory diseases, individually or for groups of patients.

8. A procedure for the generation of dry aerosols with therapeutic effect, according to claim 4, characterised by the fact that the aerosols obtained with grain dimensions of 0.3-5 microns and a total number of generated particles of 2-100 mil/mc are used in therapies of short or long duration, in acute or chronic respiratory problems or in human or veterinary problems, as well as in preventive actions for the respiratory diseases, individually or for groups of patients.

9. A device for the generation of dry aerosols, with therapeutic effect, characterised by the fact that some grains with controlled structure and composition having grains between 0.1-5 mm, preferably 0.1-2 mm, are placed at the lower part of a plastic filtering cartridge with a diameter of 20-150 mm, preferably 30-40 mm, and a height of 2-4 times the diameter, through which passes an air or air-oxygen feed with an adjustable flow between 1-301/min, preferably 2-101/min, through a lower connection with a diameter of 3-10 mm, preferably 3-6 mm, equipped with a sieve, assuring the average stirring of the grain layer over which there is a free area, while over another perforated plastic sieve, between which there is a filtering material, the air feed passes through another layer with a three-dimensional spongy structure made of saline grains with a grain of 4-6 mm, that assures supplementary air treatment, by filtering and ionization, air that afterwards is sent enriched in dry aerosols, through a connection in the environment or to a breathing mask or to a hose that goes to a breathing mask.
